(19) United States
(12) Patent Application Publication
(10) Pub. No.: US 2006/0222712 A1
(43) Pub. Date: Oct. 5, 2006

(54) Acne gel

(75) Inventors: Lincoln Krochmal, Los Gatos, CA (US); Alex Yaroshinsky, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Connetics Corporation, Palo Alto, CA (US)

(21) Appl. No.: 11/356,516

(22) Filed: Feb. 17, 2006

Related U.S. Application Data
(60) Provisional application No. 60/654,372, filed on Feb. 17, 2005.

(51) Int. Cl. A61K 31/7034; A61K 31/203; A61K 9/14

(52) U.S. Cl. 424/486; 514/35; 514/559

(57) ABSTRACT

This combination of clindamycin (1%) and tretinoin (0.025%), solubilized in a hydrogel, resulted in significantly greater improvements in acne vulgaris (reduced lesion counts and ISGA) than either drug alone or vehicle and effectively treated both non-inflammatory and inflammatory lesions with a convenient, once-daily application.

### CROSS-REFERENCES TO RELATED APPLICATIONS

This application claims priority to U.S. Provisional Application No. 60/654,372, filed Feb. 17, 2005, the teaching of which is hereby incorporated by reference in its entirety.

### BACKGROUND OF THE INVENTION

Acne is a multi-factorial disease of the pilosebaceous unit, involving bacterial colonization, local inflammation, and abnormalities in follicular keratinization and sebum production (see, Leyden, J. J., J Am Acad Dermatol; 49:S200-10 (2003); Lever, L., Marks, R., Drugs, 39(5):681-692 (1990)). Acne vulgaris is most common in late adolescence and early adulthood. This population of patients generally has a low compliance with treatment regimens unless response to therapy is rapid and noticeable (see, Draelos Z K, J Am Acad Dermatol, 32:S42-48 (1995)). The frequency and severity of adverse effects, specifically application site irritation, affects patient compliance with topical acne treatments.

Clindamycin is a topical antibiotic indicated for the treatment of acne and improves symptoms of acne by reducing levels of P. acnes and decreasing inflammation (see, Webster, G. F. et al., Antimicrob Agents Chemother, 21:770-772 (1982); Leyden, J. J., Cutis, 49:8-11 (1992)). Topical tretinoin is a retinoid indicated for the treatment of acne, affecting acne by normalizing follicular keratinization and slowing the desquamation process (see, Kligman, A. M. et al., Acgta Dermato-Venereologica (Stockholm), [Suppl. 74]:111-115 (1975); Bergfeld, W. F., J Drug Dev Clin Pract, 8:151-60 (1996); Chalker, D. K., et al., J Am Acad Dermatol, 17:251-254 (1987)). Tretinoin is also commonly associated with skin irritation. Both clindamycin and tretinoin are frequently prescribed for separate application in order to combat multiple pathogenic factors of acne vulgaris.

In a recent consensus conference, the combination of a topical retinoid and an antibiotic were recommended for the treatment of acne in a majority of patients (see, Gollnick H. et al., J Am Acad Dermatol, 49:S1-38 (2003)). It has been suggested that the drug vehicle can play a role in the degree of skin irritation (see, Piacquadio, D. and Kligman, A., J Am Acad Dermatol, 39:S67-73 (1998)). Combining clindamycin and tretinoin in the treatment of acne has required separate application regimens due to drug and vehicle incompatibilities. The surprising development of a hydrogel to stabilize clindamycin and tretinoin in one formulation at room temperature provides a convenient, once-daily treatment for acne.

### BRIEF SUMMARY OF THE INVENTION

In one embodiment, the present invention provides a method for treating non-inflammatory lesions of acne vulgaris, comprising the step of administering a composition comprising clindamycin or a pharmaceutically acceptable salt or a prodrug thereof to a subject, to treat the non-inflammatory lesions.

In another embodiment, the composition comprises clindamycin phosphate. In a further embodiment, the composition contains clindamycin phosphate at 0.25% to about 3% w/w. In still another embodiment, the composition contains clindamycin phosphate at 1.0% w/w. In yet another embodiment, the composition is a hydrogel formulation. In other embodiments, the composition is administered once daily.

In yet another embodiment, the present invention provides a method for treating inflammatory lesions of acne vulgaris, comprising administering a composition comprising tretinoin to a subject, to treat the inflammatory lesions.

In a further embodiment, the composition comprises about 0.01% to about 1% tretinoin. In still another embodiment, the composition comprises about 0.025% tretinoin. In yet another embodiment, the composition is a hydrogel formulation. In other embodiments, the composition is administered once daily.

In still yet another embodiment, the present invention provides a method for treating acne vulgaris, comprising administering a composition comprising a combination of active agents comprising clindamycin and tretinoin, wherein the median time to 50% reduction of total lesion count is shorter than for either active agent alone.

In another embodiment, the composition comprises clindamycin phosphate. In a further embodiment, the composition contains clindamycin phosphate at 0.25% to about 3% w/w. In still another embodiment, the composition contains clindamycin phosphate at 1.0% w/w. In a further embodiment, the composition comprises about 0.01% to about 1% tretinoin. In still another embodiment, the composition comprises about 0.025% tretinoin. In yet another embodiment, the composition is a hydrogel formulation. In other embodiments, the composition is administered once daily. In still other embodiments, the composition treats both inflammatory and non-inflammatory lesions.

In another embodiment, the present invention provides a use of a composition comprising clindamycin or a pharmaceutically acceptable salt or a prodrug thereof in the manufacture of a medicament for treating non-inflammatory lesions of acne vulgaris.

In still another embodiment, the present invention provides a use of a composition comprising tretinoin in the manufacture of a medicament for treating inflammatory lesions of acne vulgaris.

In yet another embodiment, the present invention provides a use of a composition comprising a combination of active agents comprising clindamycin and tretinoin in the manufacture of a medicament for treating acne vulgaris wherein the median time to 50% reduction of total lesion count is shorter than for either active agent alone.

These and other aspects, embodiments and features will become more apparent when read with the detailed description and figures which follow.

### BRIEF DESCRIPTION OF THE DRAWINGS

FIG. 1 illustrates a graph showing percentage of subjects with at least a 50% reduction in total lesion counts for the combination, clindamycin, tretinoin and vehicle composition at 2, 4, 8 and 12 weeks.

FIG. 2 illustrates a graph showing the combination composition having the fastest median time to achieve 50% reduction in total lesions as compared to the clindamycin, tretinoin and vehicle compositions.

FIG. 3 illustrates a graph showing combination gel having more subjects with clear or almost clear skin at treatments end as compared to the clindamycin, tretinoin and vehicle compositions.

FIG. 4 illustrates a graph showing the percentage of subjects with treatment-related adverse experiences.

FIG. 5 illustrates a graph showing percentage of subjects that experienced treatment-related adverse effects for the combination, clindamycin, tretinoin and vehicle compositions.

FIG. 6 illustrates a graph showing the mean percent reduction in inflammatory lesion counts in the ITT population for the combination, clindamycin, tretinoin and vehicle compositions.

FIG. 7 illustrates a graph showing the mean percent reduction in non-inflammatory lesion counts in the ITT population for the combination, clindamycin, tretinoin and vehicle compositions.

FIG. 8 illustrates a graph showing the percent of subjects with clear or almost clear skin at week 12 according to the Investigator's Static Global Assessment in the ITT population for the combination, clindamycin, tretinoin and vehicle compositions.

FIG. 9 illustrates a graph showing the mean percent reduction in total lesion counts for the combination, clindamycin, tretinoin and vehicle compositions.

### DETAILED DESCRIPTION OF THE INVENTION

### I. Definitions

As used herein, the term “lesion” refers to, for example, pustules, papules, open and closed comedones and nodules. Inflammatory lesions include, but are not limited to, pustules, papules and nodules. Non-inflammatory lesions include, but are not limited to, open and closed comedones. One of skill in the art will recognize that the methods of the present invention are useful for treating other types of lesions of acne vulgaris.

As used herein, the term “treat” or “treating” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom or condition. The treatment or amelioration of symptoms can be based on any objective or subjective parameter; including, e.g., the result of a physical examination. For example, the methods of the invention selectively treat acne vulgaris by reducing the number of inflammatory and non-inflammatory lesions.

As used herein, the term “hydrogel” refers to a highly-interdependent, biphasic matrix consisting of a solid component (usually a polymer, and more commonly a highly cross-linked polymer) that has both hydrophilic and hydrophobic character. Additionally, the matrix has a liquid component (e.g., water) that is retained in the matrix by intermolecular forces. The hydrophobic character provides the matrix with a degree of water insolubility while the hydrophilic character affords water permeability. One of skill in the art will appreciate that several different types of polymers can be used in combination to form hydrogels useful in the methods of the present invention. The hydrogel disclosed in U.S. Pat. No. 5,690,923, incorporated herein by reference, is particularly preferred.

As used herein, the term “a combination of active agents” refers to a composition of at least two active agents. In the present invention, a combination of active agents can include clindamycin and tretinoin. Other active agents are also useful in the methods of the present invention.

As used herein, the term “50% reduction of total lesion count” refers to a reduction of at least 50% in the total count of inflammatory and non-inflammatory lesions.

As used herein, the term “salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (chloride, sulfate, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

As used herein, the term “prodrug” refers to covalently bonded carriers which are capable of releasing the active agent of the methods of the present invention, when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of the active agents of the present invention include active agents wherein a hydroxy, amidino, guanidino, amino, carboxylic or a similar group is modified.

### II. Methods

The methods of the present invention are useful for the treatment of non-inflammatory and inflammatory lesions of acne vulgaris using clindamycin or tretinoin, either alone or in combination. The acne vulgaris is treated by administering to the subject a composition comprising an active agent alone, or a combination of active agents.

A. Administration

The compositions of the present invention can be administered at a variety of intervals. In some instances, administration once a day is preferable. In other instances, administration can be less or more frequently, such as 1, 2, 3, or 4 times a day, 1 time every 2 days, or once a week.

B. Subject Evaluation

In certain aspects, the methods of the present invention include evaluating each subject for the number of lesions, providing an overall assessment of the subject's facial acne using the Investigator's Static Global Assessment, further evaluation of additional signs of acne, and finally an evaluation of some symptoms.

The first step is the counting of lesions. Lesions that are included in the count can include inflammatory as well as non-inflammatory lesions. The count of lesions can be preferably performed at each study visit using the following guidelines: (1) preferably, the same person should perform all of the lesion counts for a given subject as they progress through the study to ensure consistency; (2) preferably, the count includes only the face in these assessments, the face is defined as the hairline edge to the mandibular line; and (3) does not count non-inflammatory lesions (comedones) on the nose.

At each study visit, the investigator/designee will provide an overall assessment of the subject's facial acne vulgaris using the Investigator's Static Global Assessment using the following scale:
Grade 0=Normal, clear skin with no evidence of acne vulgaris
Grade 1=Skin almost clear: rare non-inflammatory lesions present, with rare non inflamed papules (papules must be resolving and may be hyper-pigmented, though not pink-red) requiring no further treatment in the Investigator's opinion
Grade 2=Some non-inflammatory lesions are present, with few inflammatory lesions (papules/pustules only, no nodulo-cystic lesions)
Grade 3=Non-inflammatory lesions predominate, with multiple inflammatory lesions evident: several to many comedones and papules/pustules, and there may or may not be 1 small nodulo-cystic lesion
Grade 4=Inflammatory lesions are more apparent: many comedones and papules/pustules, there may or may not be a few nodulo-cystic lesions
Grade 5=Highly inflammatory lesions predominate: variable number of comedones, many papules/pustules and nodulo-cystic lesions

Signs of acne vulgaris, such as scaling, dryness, and erythema, are evaluated. The severity of each sign can be graded by the investigator/designee based on the appearance at each study visit using the following scale:

None (0)=Normal

Trace (1)=Mild and localized

Mild (2)=Mild and diffuse

Moderate (3)=Moderate and diffuse

Marked (4)=Moderate and dense

Severe (5)=Prominent and dense

Additional symptoms such as burning and itching can also be evaluated. The severity of a symptom will be graded based upon the subject's impression during the last week using the following scale:
None (0)=Normal, no discomfort
Trace (1)=An awareness, but no discomfort and no intervention required
Mild (2)=A noticeable discomfort that causes intermittent awareness
Moderate (3)=A noticeable discomfort that causes continuous awareness
Marked (4)=A definite discomfort that causes continuous awareness and interferes occasionally with normal daily activities
Severe (5)=A definite continuous discomfort that interferes with normal daily activities

C. Treatment

In certain other aspects, the present invention provides a method for treating a skin disorder in a subject, comprising administering a hydrophilic composition to an affected area of the subject's skin having such a skin disorder in an amount and for a period of time sufficient to improve the skin disorder. Preferably, the composition is administered once a day over the treatment period. Depending on the patient's improvement, the treatment can be extended for less than a week, as well as up to two months or more. Certain treatment periods include, for example, 1 week to 15 weeks, such as 1, 2, 3, 4, 5, 6, 7, 8, 9, 10, 11, 12, 13, 14 and 15 week periods. The progress of improvement can be monitored by the subject, by a healthcare provider or both. The treatment period can for example be about 8 weeks.

A wide variety of skin disorders are suitable for treatment using the present methods, uses and compositions of the present invention. These skin disorders include, but are not limited to, acne vulgaris, rosacea, and various inflammatory or non-inflammatory conditions including atopic dermatitis. In certain aspects, the skin disorder is a non-inflammatory lesion form of acne vulgaris, an inflammatory lesion form of acne vulgaris, acne vulgaris per se or a combination thereof.

The affected area of the subject's skin can be anywhere on the body in which the skin disorder exists. Preferably, the hydrogel composition is administered to the face. The amount of composition and period of administration time sufficient to improve the skin disorder will be dependent on the subject, the skin condition and the severity of the condition. Generally, a sufficient amount will be applied directly on the affected area.

### III. Compositions

In certain other aspects, the compositions of the present invention comprise clindamycin or tretinoin separately or in combination. Other active agents are also useful in the compositions of the present invention and will be recognized by one of skill in the art.

Clindamycin can be used in its neutral form, or in its salt form. Salt forms include for example, clindamycin phosphate. One of skill in the art will appreciate that other salt forms of clindamycin are useful in the present invention.

Clindamycin is an antibiotic also known as methyl 7-chloro-6,7,8-trideoxy-6-(1-methyl-trans-4-propyl-L-2-pyrrolidinecarboxamido)-1-thio-L-threo-α-D-galacto-octo-pyranoside or methyl 7-chloro-6,7,8-trideoxy-6-[[(1-methyl-4-propyl-2-pyrrolidinyl)carbonyl]amino]-1-thio-L-threo-α-D-galacto-octo-pyranoside. As used herein, the term “clindamycin” alone includes free-base clindamycin as well as the pharmaceutically acceptable salts and esters thereof. Examples of pharmaceutically acceptable salts and esters of clindamycin include, but are not limited to, clindamycin hydrochloride, clindamycin phosphate, clindamycin palmitate, and clindamycin palmitate hydrochloride. It is preferred to use a clindamycin salt or ester in the compositions of the present invention, with clindamycin phosphate being especially preferred.

Clindamycin is present in the compositions of the present invention in an amount from about 0.25% to about 3% w/w. In some instances, clindamycin is present in an amount of about 1.0% w/w.

Tretinoin is present in the compositions of the present invention in an amount from about 0.01% to about 1.0% w/w. In some instances, tretinoin is present in an amount of about 0.025% w/w.

A hydrogel is a highly-interdependent, biphasic matrix consisting of a solid component (usually a polymer, and more commonly a highly cross-linked polymer) that has both hydrophilic and hydrophobic character. Additionally, the matrix has a liquid component (e.g., water) that is retained in the matrix by intermolecular forces. The hydrophobic character provides the matrix with a degree of water insolubility while the hydrophilic character affords water permeability.

The polymer portion of the hydrogel will contain functionality which is suitable for hydrogen bonding (e.g., hydroxyl groups, amino groups, ether linkages, carboxylic acids and esters, and the like). Moreover, the affinity for water presented by the hydrogen bonding functionality must be of sufficient degree that the hydrated hydrogel will retain the water within its matrix even upon placement of the hydrogel in a hydrophobic medium such as an oil or lipid matrix. In addition to this binding of water within the hydrogel matrix, the hydrogel should allow water to flow through it when placed in an aqueous environment. A number of hydrogels have been developed for use as contact lenses. These hydrogels keep a layer of water at the surface of the eye to protect the eye from drying out.

Examples of a hydrophilic polymer useful in the present invention includes, but is not limited to, polyethylene glycol (for instance, Macrogol 400, Macrogol 1500, Macrogol 4000, Macrogol 6000, and Macrogol 20000 (all made by Nihon Yushi)), polyvinyl pyrrolidone (for instance, water-soluble polymers such as PVP™ K30 (BASF)), sugar alcohols, such as D-sorbitol and xylitol, saccharides such as sucrose, maltose, lactulose, D-fructose, dextran (for instance, Dextran 40), and glucose, surfactants such as polyoxyethylene hydrogenated castor oil (for instance, Cremophor™ RH40 (BASF) HCO-40, HCO-60 (Nikko Chemicals), polyoxyethylene polyoxypropylene glycol (for instance, Pluronic™ F68 (Asahi Denka), etc.) or polyoxyethylene sorbitan higher fatty acid esters (such as Tween 80 (Kanto Kagaku (Chemical)), etc.), salts, such as sodium chloride and magnesium chloride, organic acids such as citric acid and tartaric acid, amino acids such as glycine, β-aniline, lysine hydrochloride, and amino saccharides such as meglumine, etc. Polyethylene glycol, sucrose and polyvinyl pyrrolidone are preferred and polyethylene glycol (particularly Macrogol 6000) is further preferred. Moreover, one or a combination of 2 or more hydrophilic bases can be used in the present invention.

When the hydrophilic base is added in the present invention, the ratio used is preferably 1 to 80 wt % per total preparation, particularly 5 to 60 wt %, per total preparation.

Other additives that are pharmaceutically acceptable can be added as needed to the pharmaceutical composition of the present invention. For instance, one or a combination of two or more of fillers such as lactose, mannitol, potato starch, wheat starch, rice starch, corn starch, crystalline cellulose, methyl cellulose, gum Arabic, etc., viscosity-increasing agents, such as carboxymethyl cellulose, carboxymethyl cellulose calcium, carboxymethyl cellulose, etc., lubricants such as stearic acid, calcium stearate, magnesium stearate, talc, magnesium metasilicoaluminate, calcium hydrogen phosphate, anhydrous calcium hydrogen phosphate, etc., fluidizers such as silicon dioxide hydrate, light silic anhydride, dry aluminum hydroxide, etc., surfactants such as sodium laurylsulfate sucrose fatty acid esters, etc., coating agents such as zein, hydroxypropyl methyl cellulose, hydroxypropyl cellulose, etc., flavorings such as 1-menthol, mentha oil, fennel oil, etc., preservatives such as sodium sorbate, potassium sorbate, methyl parahydroxybenzoate, ethyl parahydroxybenzoate, etc., buffers such as citric acid, succinic acid, glycine, aspartic acid, alanine, arginine and its salts, magnesium oxide, zinc oxide, magnesium hydroxide, phosphoric acid, boric acid its salts, etc., can be added as needed.

### IV. Examples

### Example 1

### Time to Response with Combination Therapy Versus Single Agents for Inflammatory and Non-Inflammatory Lesions of Acne Vulgaris

In these two studies, the time to response with the combination hydrogel was compared with each agent alone and with the vehicle. The results of these two studies demonstrate that a standardized, once-daily dosing regimen of the combination of clindamycin (1%) and tretinoin (0.025%), solubilized in a hydrogel provide significantly faster improvement in acne vulgaris (8 weeks to a 50% reduction in total lesion counts) than clindamycin (12 weeks), tretinoin (12 weeks) or the vehicle (not reached by end of trial).

Methods

Design: These two 12-week studies were randomized, double-blind, active- and vehicle-controlled, multicenter clinical studies.

Participants: Men and women aged 12 years or older with 17-40 facial inflammatory lesions (papules plus pustules) including nasal lesions and 20-150 facial non-inflammatory lesions (open and closed comedones), excluding nasal lesions, Table 1. Baseline demographics were not statistically different between the four treatment groups (Table 1).

Treatments: Participants were randomized to a once-daily application of:
Combination clindamycin (1%) and tretinoin (0.025%) hydrogel for 12 weeks, or
Clindamycin (1%) hydrogel for 12 weeks, or
Tretinoin (0.025%) hydrogel for 12 weeks, or
Vehicle hydrogel for 12 weeks

Data collection: Efficacy and safety data were collected at Baseline, Week 2, Week 4, Week 8, and Week 12, based on vital signs, signs and symptoms of facial acne and adverse experiences reported by the subjects.

Endpoints: The primary endpoints were the percent reduction in inflammatory, non-inflammatory, and total lesion counts from Baseline to Week 12 (end of treatment with last observation carried forward) and the proportion of subjects who had an Investigator's Static Global Assessment (ISGA) score of 0 or 1 at Week 12 (end of treatment). The secondary endpoint was the time to a 50% reduction in lesion counts. If 12-week data were not available, the last observation was carried forward (LOCF).

TABLE 1
Baseline demographics

| | Combination | Clindamycin | Tretinoin | Vehicle |
|---|---|---|---|---|
| Number of | 634 | 635 | 635 | 315 |
| Subjects | | | | |
| Age | | | | |
| Mean | 19.3 (12-49) | 19.6 (11-54) | 20.2 (1-81) | 19.0 (12-56) |
| (y; range) | | | | |
| ≦16 years | 328 (52%) | 301 (47%) | 284 (45%) | 169 (54%) |
| >16 years | 306 (48%) | 334 (53%) | 351 (55%) | 146 (46%) |
| Sex | | | | |
| Male | 296 (47%) | 297 (47%) | 266 (42%) | 164 (52%) |
| Female | 338 (53%) | 338 (53%) | 369 (58%) | 151 (48%) |
| Race | | | | |
| Asian | 11 (2%) | 18 (3%) | 15 (2%) | 9 (3%) |
| Caucasian | 425 (67%) | 406 (64%) | 409 (64%) | 202 (64%) |
| African- | 118 (19%) | 123 (19%) | 131 (21%) | 65 (21%) |
| American | | | | |
| Hispanic | 65 (10%) | 64 (10%) | 58 (9%) | 31 (10%) |
| Other | 15 (2%) | 24 (4%) | 22 (3%) | 8 (3%) |
| Lesion counts | | | | |
| (mean; std) | | | | |
| Total | 78.3 (32.2) | 76.4 (30.5) | 76.7 (30.5) | 78.1 (31.2) |
| Inflammatory | 26.3 (7.4) | 26.2 (7.5) | 26.0 (7.2) | 26.4 (7.0) |
| Non- | 52.0 (28.8) | 50.2 (28.2) | 50.7 (27.7) | 51.7 (29.3) |
| inflammatory | | | | |

Results

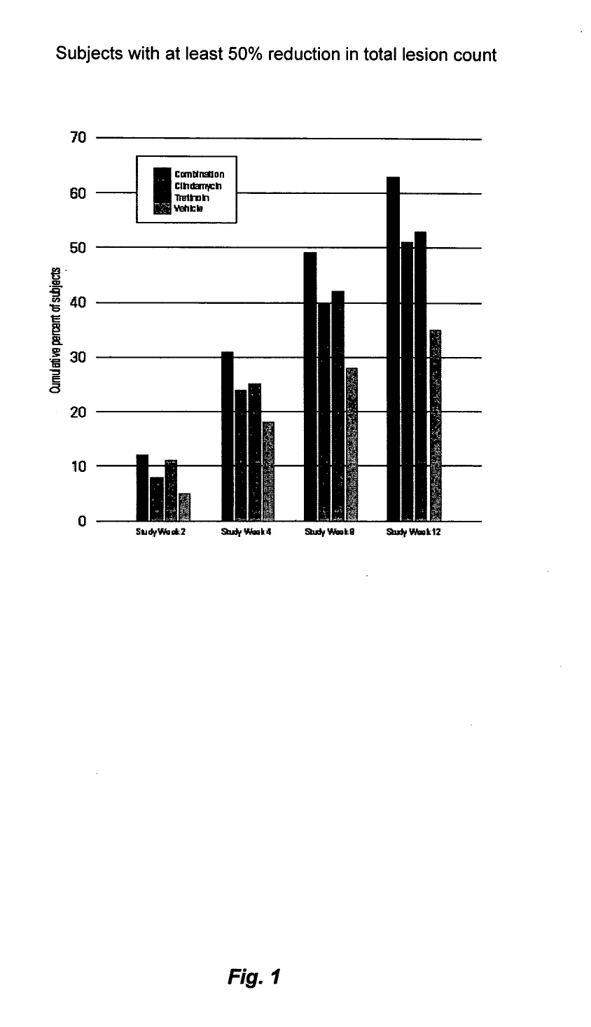

The study enrolled 2219 subjects. The mean age was 20, and about half were younger than 17 years of age. At baseline, patients had a mean of 26 inflammatory lesions and approximately 51 non-inflammatory lesions. Baseline characteristics across the four treatment groups were not statistically different. At each time point, a higher percentage of patients in the combination group had a 50% reduction in comparison with the other three treatment groups (FIG. 1).

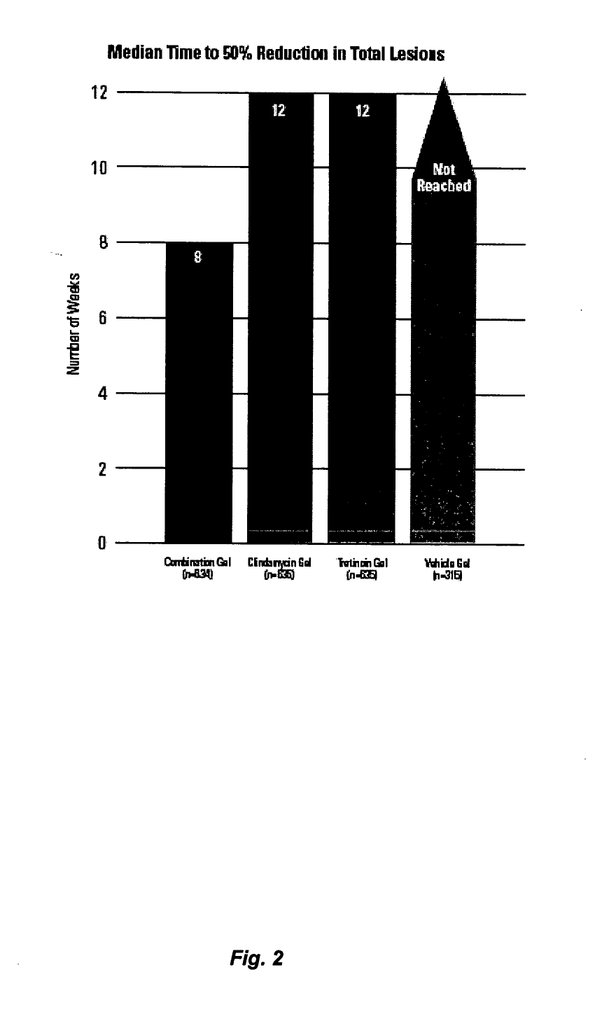

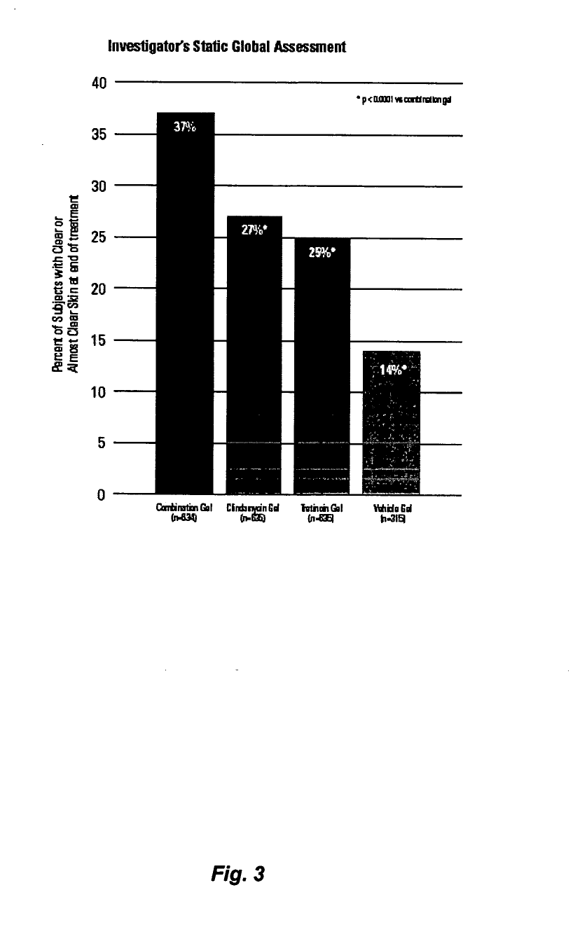

The median time to 50% reduction in total lesion counts was 8 weeks with combination, which was significantly faster than with clindamycin (12 weeks; p<0.0001), tretinoin (12 weeks; p<0.001), and vehicle (p<0.0001; FIG. 2). From Baseline to Week 12 (end of treatment), the percentage reduction in inflammatory and noninflammatory lesion counts with the combination was significantly higher than with each of the other three 15 treatments (p<0.004). The percentage of subjects with clear or almost clear skin on the ISGA was significantly higher in the combination group than in the other three treatment groups (p<0.0002; FIG. 3).

### Example 2

### Tolerability Assessment of Combination Clindamycin/Tretinoin Hydrogel for the Treatment of Acne Vulgaris in 2219 Subjects

Two studies were performed to compare the tolerability (and efficacy) of a combination clindamycin (1%) and tretinoin (0.025%) hydrogel with each agent alone and with the vehicle in subjects with mild to moderate acne vulgaris. These two randomized, double-blind trials demonstrate that the combination of tretinoin and clindamycin as formulated in the hydrogel are well tolerated. The active controls, tretinoin alone in hydrogel and clindamycin alone in hydrogel, as well as the hydrogel vehicle itself are also well tolerated. Of the 2219 subjects in the two trials, 1902 (85.7%) completed the 12-week treatment period, and 1943 (87.6%) subjects reported no adverse experiences. Only 28 of 2219 subjects (1.3%) withdrew due to an adverse experience. The incidence of application site reactions in the combination group over the 12-week treatment period was low and similar to tretinoin alone.

The methods of Example 1 were followed.

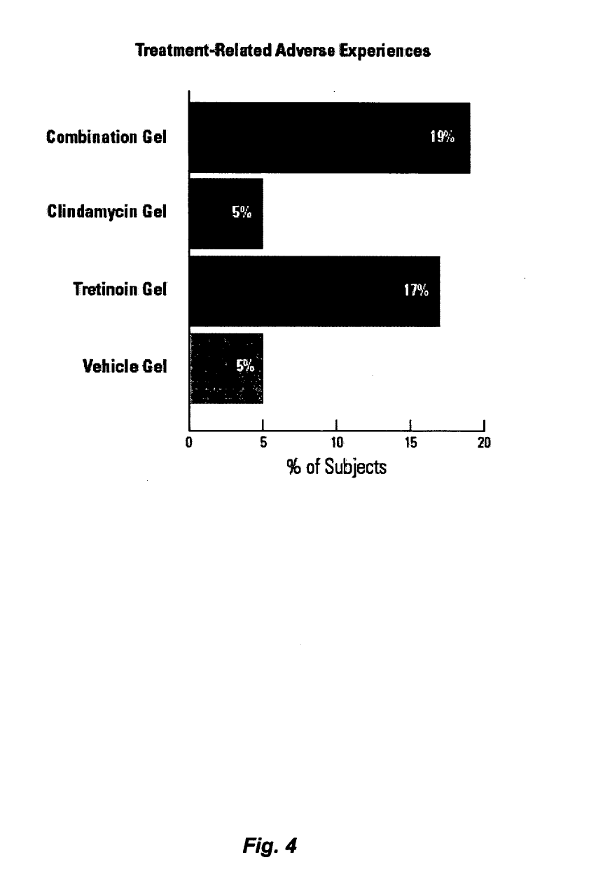

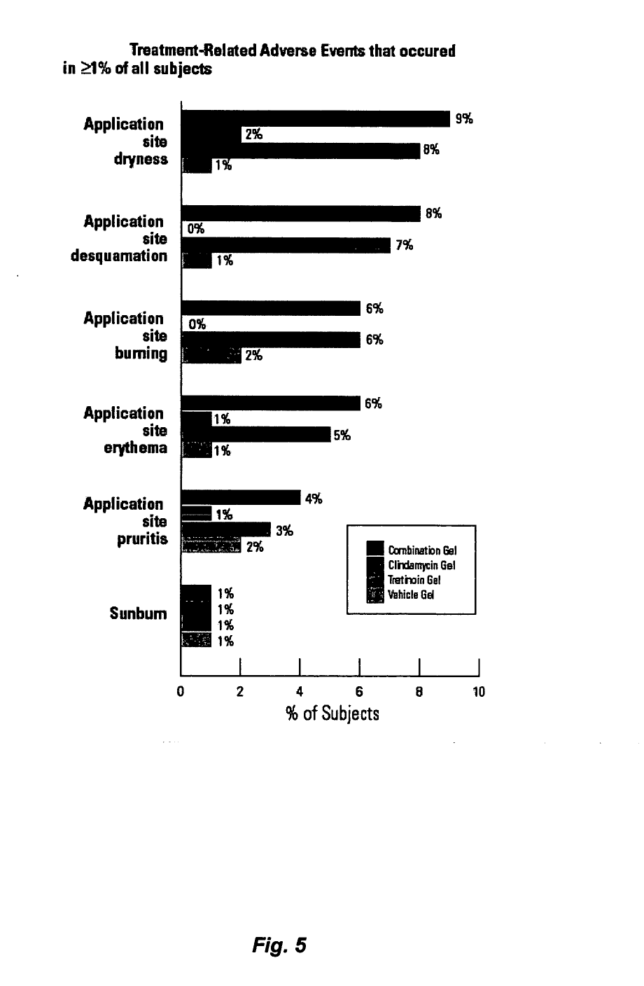

Of the 2219 subjects enrolled and randomized, 1902 (85.7%) completed the 12-week treatment period. Of those that withdrew, 28 (1.3%) subjects withdrew due to an adverse experience, 17 (60.7%) of whom were in the tretinoin group. Overall, the active treatments were well tolerated, and 1943 (87.6%) subjects reported no adverse experiences. Treatment-related adverse experiences are summarized in FIGS. 4 and 5. There were 7 (0.3%) serious adverse events, and none were related to treatment. Treatment-related adverse experiences that were considered severe occurred in the combination and tretinoin treatment groups and included application site burning (2 and 3 subjects, respectively), application site desquamation (3 and 3 subjects, respectively), application site dryness (2 and 1 subjects, respectively), application site erythema (4 and 1 subjects, respectively), application site pruritus (1 and 3 subjects, respectively), application site rash (0 and 1 subjects, respectively), application site swelling (1 and 0 subjects, respectively), and application site vesicles (1 and 0 subjects, respectively). Combination was significantly better at reducing inflammatory and non-inflammatory lesion counts than each of the other three treatments (p<0.005 & p<0.004, respectively). The combination group had a significantly higher proportion of subjects (37%) scoring 0 or 1 on the ISGA at Week 12 than with clindamycin (27%), tretinoin (25%) or vehicle (14%; p<0.0001 for each comparison).

The three active treatments evaluated in this study were well tolerated, with 87.6% of subjects showing no adverse experiences at all. The treatment-related adverse events in the combination, clindamycin, tretinoin and vehicle treatment groups and the total study group were application site dryness (9%, 2%, 8%, 1%, and 5%, respectively), application site desquamation (8%, 0%, 7%, 1%, and 5%, respectively), application site burning (6%, 0%, 6%, 2%, and 4%, respectively) application site erythema (6%, 1%, 5%, 1%, and 3%, respectively), application site pruritus (4%, 1%, 3%, 2%, and 3%, respectively), and sunburn (1%, 1%, 1%, 1%, and 1%, respectively). Overall, less than 1.5% (28/2219) of participants discontinued treatment due to adverse events. The 12-week treatment period was completed by 1902 subjects (85.7%).

TABLE 2
Incidence of treatment-related adverse experiences in ≧1% of subjects

| | Combination | Clindamycin | Tretinoin | Vehicle | |
|---|---|---|---|---|---|
| | Gel | Gel | Gel | Gel | Total |
| Number of Subjects | 634 | 635 | 635 | 315 | 2219 |
| Application site dryness | 57 (9%) | 12 (2%) | 48 (8%) | 3 (1%) | 120 (5%) |
| Application site desquamation | 51 (8%) | 2 (0%) | 47 (7%) | 2 (1%) | 102 (5%) |
| Application site burning | 40 (6%) | 3 (0%) | 41 (6%) | 5 (2%) | 89 (4%) |
| Application site erythema | 35 (6%) | 6 (1%) | 33 (5%) | 3 (1%) | 77 (3%) |
| Application site pruritus | 24 (4%) | 7 (1%) | 21 (3%) | 6 (2%) | 58 (3%) |
| Sunburn | 9 (1%) | 5 (1%) | 5 (1%) | 3 (1%) | 22 (1%) |

### Example 3

### Two Randomized, Controlled Trials of a Combination Clindamycin/Tretinoin Hydrogel Compared with each Agent Alone for the Treatment of Acne Vulgaris In 2219 Subjects

These two studies compare the efficacy of the combination of clindamycin (1%) and tretinoin (0.025%) in hydrogel with each agent alone and vehicle in subjects with Grade 2-3 acne vulgaris.

The data demonstrates that the combination is effective in treating both inflammatory and non-inflammatory lesions. Overall, the incidence of irritation and application site reactions in the combination group is low and similar to tretinoin alone. This study demonstrates that the combination of clindamycin and tretinoin, solubilized in a hydrogel, results in significantly greater improvements in acne vulgaris than either drug alone or vehicle.

The methods of Example 1 were followed.

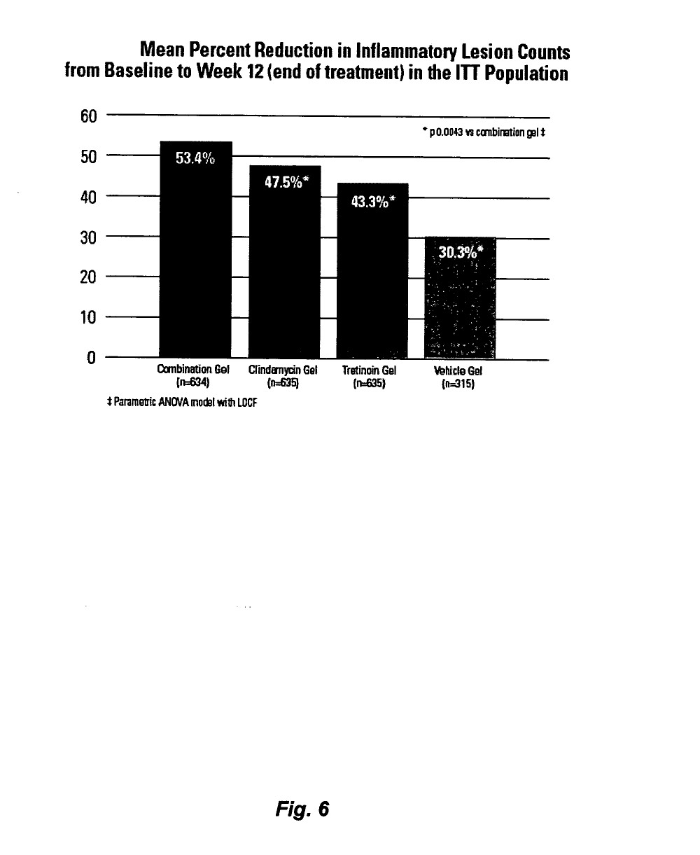

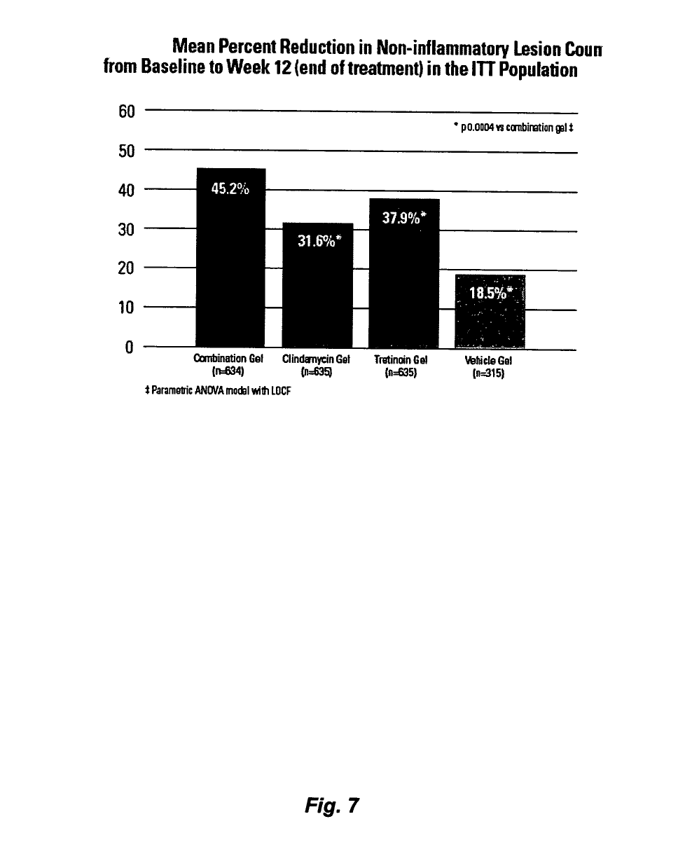

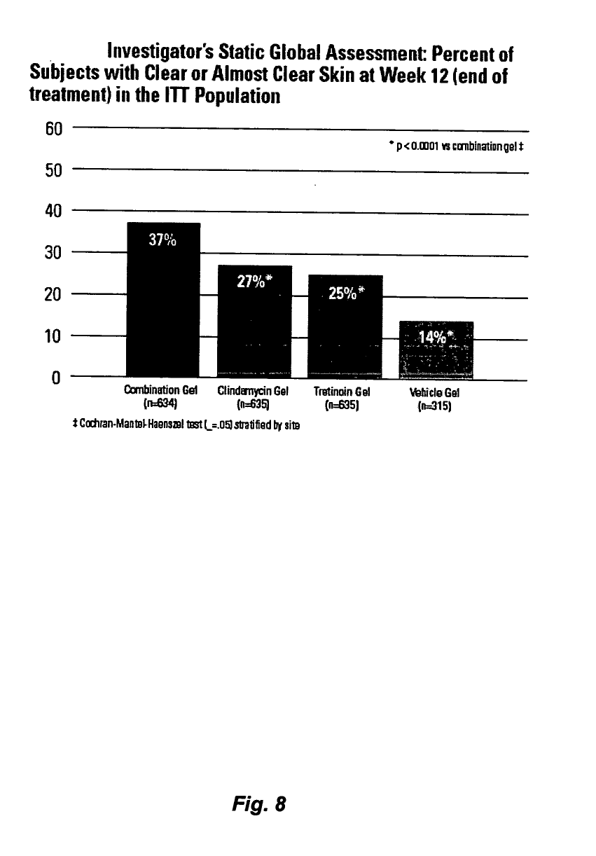

From Baseline to Week 12, the percent reduction in inflammatory lesions (FIG. 6) and non-inflammatory lesions (FIG. 7) was significantly greater with the combination than with each active alone or the vehicle. At the end of treatment, 37% of subjects treated with the combination had clear or almost clear skin requiring no further treatment as assessed with the ISGA (FIG. 8).

Adverse Effects. The three active treatments evaluated in this study were well tolerated, with 87.6% of subjects showing no adverse experiences at all (Table 2). Overall, less than 1.5% (28/2219) of participants discontinued treatment due to adverse events. None of the 7 serious adverse events were treatment-related. The 12-week treatment period was completed by 1901 (85.7%) subjects.

### Example 4

### Acne Treatment Outcomes with a Once-Daily Combination of Clindamycin and Tretinoin in Hydrogel Compared to Single Agents

The objective of this study was to compare the efficacy and safety of a novel combination of clindamycin (1%) and tretinoin (0.025%) in hydrogel with each agent alone and with the vehicle for the treatment of mild-to-moderate acne vulgaris.

These data demonstrate that a standardized, once-daily dosing regimen of the combination of clindamycin (1%) and tretinoin (0.025%), solubilized in a hydrogel formulation provides significant improvement in acne severity as compared with monotherapy. Once-daily application of the combination may improve compliance over separate application of the two drugs.

The methods of Example 1 were followed.

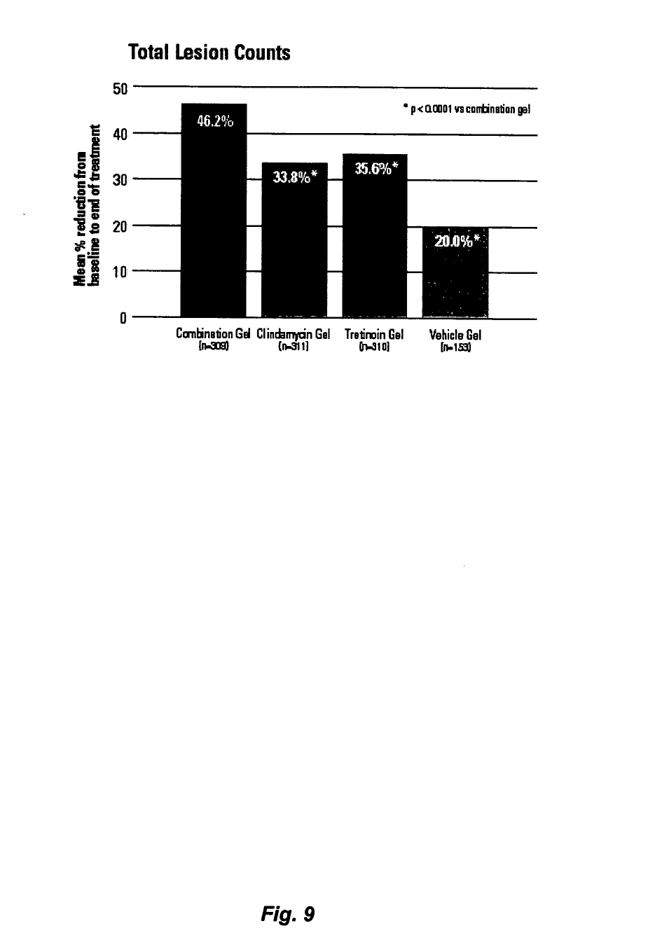

This study randomized 1083 subjects including 309 on combination, 311 on clindamycin, 310 on tretinoin, and 153 on vehicle. The mean age was 20 and about half were younger than 17 years of age. About half the subjects were female. From baseline to week 12, the percent reduction in lesion counts was significantly greater with the combination of clindamycin and tretinoin (46.2%) than with either clindamycin (33.8%) or tretinoin (35.6%) alone, or the vehicle (20.0%; p<0.0001 for each comparison) as shown in FIG. 9. The percentage of subjects who met the criteria for success on the ISGA (clear or almost clear skin) in the combination group (35%) was significantly higher than with clindamycin (21%; p=0.0002), tretinoin (19%; p<0.0001) or vehicle (12%; p<0.0001).

Adverse Effects. Overall, the combination was well tolerated, and the incidence of adverse experiences with combination was similar to tretinoin alone and generally higher than with clindamycin alone (Table 3). The 12-week treatment period was completed by 909 participants (84%), and less than 1.5% (16/1083) of participants discontinued treatment due to adverse experiences.

TABLE 3
Incidence of treatment-related adverse experiences in ≧1% of the ITT population
(n = 1083)

| | Combination | Clindamycin | Tretinoin | Vehicle | |
|---|---|---|---|---|---|
| | Gel | Gel | Gel | Gel | Total |
| Number of Subjects | 309 | 311 | 310 | 153 | 1083 |
| Application site burning | 17 (6%) | 1 (0%) | 25 (8%) | 0 | 43 (4%) |
| Application site desquamation | 19 (6%) | 2 (1%) | 23 (7%) | 1 (1%) | 45 (4%) |
| Application site dryness | 24 (8%) | 7 (2%) | 24 (8%) | 0 | 55 (5%) |
| Application site erythema | 14 (5%) | 3 (1%) | 20 (6%) | 0 | 37 (3%) |
| Application site irritation | 4 (1%) | 1 (0%) | 2 (1%) | 0 | 7 (1%) |
| Application site pruritus | 11 (4%) | 2 (1%) | 10 (3%) | 1 (1%) | 24 (2%) |
| Application site rash | 2 (1%) | 0 | 5 (2%) | 0 | 7 (1%) |
| Sunburn | 6 (2%) | 1 (0%) | 2 (1%) | 1 (1%) | 10 (1%) |

At the end of treatment, 35% of subjects treated with the combination had clear or almost clear skin requiring no further treatment as assessed with the ISGA. These data demonstrate that the combination is effective in treating both inflammatory and non-inflammatory lesions. Overall, the incidence of irritation and application site reactions in the combination and tretinoin alone groups over the 12-week treatment period was low.

### Example 5

### The Combination Clindamycin/Tretinoin Hydrogel: a Randomized, Double-Blind, Active- and Vehicle-Controlled Study

The objective of this study was to compare the efficacy and safety of a combination clindamycin (1%) and tretinoin (0.025%) hydrogel with each agent alone and with the vehicle. This combination of clindamycin (1%) and tretinoin (0.025%), solubilized in a hydrogel formulation, resulted in significantly greater improvements in acne vulgaris (reduced lesion counts and ISGA) than either drug alone or vehicle and effectively treated both non-inflammatory and inflammatory lesions with a convenient, once-daily application.

This study randomized 1136 subjects including 325 on combination, 324 on clindamycin, 325 on tretinoin, and 162 on vehicle. The mean age was 19 and 50.2% were younger than 17 years of age. About half (47.7%) of the subjects were male.

The methods of Example 1 were followed.

From Baseline to Week 12 (end of treatment), the percentage reduction of inflammatory and non-inflammatory lesion counts in the combination treatment group was significantly higher than in each of the other three treatment groups (See FIGS. 11 and 12). At the end of treatment, 39% of subjects treated with the combination had clear or almost clear skin requiring no further treatment as assessed with the ISGA. This compares favorably with the percentage of subjects (32% on clindamycin, 31% on tretinoin, and 15% on vehicle) who had this result.

Adverse Effects. The three active treatments evaluated in this study were well tolerated, with 64% of participants showing no adverse experiences. The most common adverse events were application site reactions, which occurred with similar frequency in the combination and tretinoin treatment groups. Less than 1.1% (12/1136) of participants discontinued treatment due to adverse experiences. The 12-week treatment period was completed by 993 participants (87%).

TABLE 4
Incidence of treatment-related adverse experiences in ≧1% of subjects

| | Combination | Clindamycin | Tretinoin | Vehicle | |
|---|---|---|---|---|---|
| | Gel | Gel | Gel | Gel | Total |
| Number of Subjects | 325 | 324 | 325 | 162 | 1136 |
| Application site burning | 23 (7%) | 2 (1%) | 16 (5%) | 5 (3%) | 46 (4%) |
| Application site desquamation | 32 (10%) | 0 | 24 (7%) | 1 (1%) | 57 (5%) |
| Application site dryness | 33 (10%) | 5 (2%) | 24 (7%) | 3 (2%) | 65 (6%) |
| Application site erythema | 21 (6%) | 3 (1%) | 13 (4%) | 3 (2%) | 40 (4%) |
| Application site pruritus | 13 (4%) | 5 (2%) | 11 (3%) | 5 (3%) | 34 (3%) |
| Sunburn | 3 (1%) | 4 (1%) | 3 (1%) | 2 (1%) | 12 (1%) |

The combination was effective in treating both inflammatory and non-inflammatory lesions. Twelve comparisons were made between combination and the other three treatment groups (clindamycin, tretinoin, vehicle) with regard to the four primary endpoints (inflammatory, non-inflammatory, and total lesion counts and ISGA), and all twelve were statistically significant in favor of the combination. Overall, the incidences of irritation and application site reactions in the combination and tretinoin alone groups over the 12-week treatment period were low.

### Example 6

### Hydrogel Compositions

The hydrogel compositions can be prepared by first preparing three phases of material:
I. Carbopol is added to a mixture of propylene glycol and water.
II. Laureth-4 is heated until 35°-40° C., whereafter butyl-hydroxytoluene, retinoic acid and methylhydroxybenzoate are added. The mixture is stirred until complete dissolution of the components is achieved, excluding oxygen, protected against the influence of light.
III. Clindamycin phosphate, citric acid monohydrate and tromethamine are dissolved in water under stirring and heating until 50°-60° C.

While stirring, phase II is added to phase I, and then phase III is added, protected against light at a temperature of 50°-60° C. The mixture is put under vacuum, stirred, placed under vacuum again and subsequently cooled till the temperature drops below 30° C. Nitrogen is added and the product is removed from the mixer.

Additional compositions are prepared substituting 4% Laureth 4 with (1) a mixture of 2% Laureth 4 (polyoxyethylene (4) monolauryl-ether, (POE 4)) and 2% GLYCEROX LI 5 (polyoxyethylene (15) glycerinmono-laurate; (POE 15)); (2) 4% of SYNPERONIC PE/L44; and (3) 4% of TAGAT TO.

TABLE 5
hydrogel composition

| Component | % Content |
|---|---|
| Tretinoin | 0.025 |
| Clindamycin phosphate | 1.0 |
| Disodium edetate | 0.10 |
| Butylhydroxytoluene | 0.002 |
| CARBOPOL ™ 980 | 1.0 |
| Laureth-4 (POE 4) | 4.0 |
| Tromethamine | 0.75 |
| Citric acid monohydrate | 0.15 |
| Propylene glycol | 7.5 |
| Methylhydroxybenzoate | 0.1 |
| Water till | 100.0 |

As will be apparent to those of skill in the art, various other gel formulations are within the scope of the present invention. For example, the formulation in Table 5 includes 4% POE 4. However, Laureth 4 (4%) can be substituted with a mixture of Laureth 4 (polyoxyethylene (4) monolauryl-ether, (POE 4)) and GLYCEROX L15 (polyoxyethylene (15) glycerinmono-laurate (POE 15). Various combinations are listed in Table 6.

TABLE 6
combinations of POE 4 and POE 15

| FORMULATION | % Content POE 4 | % Content POE 15 |
|---|---|---|
| Formulation 1 | 4% | 0% |
| Formulation 2 | 1% | 3% |
| Formulation 3 | 0% | 4% |
| Formulation 4 | 3% | 1% |
| Formulation 5 | 2% | 2% |
| Formulation 6 | between 0%-1% | between 4%-3% |
| Formulation 7 | between 1%-2% | between 3%-2% |
| Formulation 8 | between 2%-3% | between 2%-1% |
| Formulation 9 | between 3%-4% | between 1%-0% |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method for treating non-inflammatory lesions of acne vulgaris, said method comprising:
administering a composition comprising clindamycin or a pharmaceutically acceptable salt or a prodrug thereof to a subject, to treat said non-inflammatory lesions.

2. The method of claim 1, wherein said composition comprises clindamycin phosphate.

3. The method of claim 2, wherein said composition contains clindamycin phosphate at 0.25% to about 3% w/w.

4. The method of claim 3, wherein said composition contains clindamycin phosphate at 1.0% w/w.

5. The method of claim 1, wherein said composition is a hydrogel formulation.

6. The method of claim 1, wherein said composition is administered once daily.

7. A method for treating inflammatory lesions of acne vulgaris, said method comprising:
administering a composition comprising tretinoin to a subject, to treat said inflammatory lesions.

8. The method of claim 7, wherein said composition comprises about 0.01% to about 1% tretinoin.

9. The method of claim 8, wherein said composition comprises about 0.025% tretinoin.

10. The method of claim 7, wherein said composition is a hydrogel formulation.

11. The method of claim 7, wherein said composition is administered once daily.

12. A method for treating acne vulgaris, said method comprising:
administering a composition comprising a combination of active agents comprising clindamycin and tretinoin to a subject, wherein the median time to 50% reduction of total lesion count is shorter than for either active agent alone.

13. The method of claim 12, wherein said composition comprises clindamycin phosphate.

14. The method of claim 13, wherein said composition contains clindamycin phosphate at 1.0% w/w.

15. The method of claim 12, wherein said composition comprises about 0.01% to about 1% tretinoin.

16. The method of claim 15, wherein said composition comprises about 0.025% tretinoin.

17. The method of claim 12, wherein said composition is a hydrogel formulation.

18. The method of claim 12, wherein said composition is administered once daily.

19. The method of claim 12, wherein said composition treats both inflammatory and non-inflammatory lesions.

20. The method of claim 18, wherein said composition is administered over a period of 8 weeks.

21. The method of claim 20, wherein said subject has an Investigator's Static Global Assessment (ISGA) of clear or almost clear.
